(19) United States
(12) Patent Application Publication
(10) Pub. No.: US 2022/0031937 A1
(43) Pub. Date: Feb. 3, 2022

(54) INJECTION SYSTEM, SYRINGE, AND ADAPTER

(71) Applicant: CIRCULUS INC., Tokyo (JP)

(72) Inventors: Shigeru NEMOTO, Tokyo (JP); Yumiko FUKIKOSHI, Tokyo (JP)

(73) Assignee: CIRCULUS INC., Tokyo (JP)

(21) Appl. No.: 17/298,858

(22) PCT Filed: Dec. 6, 2019
(86) PCT No.: PCT/JP2019/047855

§ 371 Date: Jun. 1, 2021

(30) Foreign Application Priority Data
Dec. 6, 2018 (JP) 2018-229264

(51) Int. Cl. A61M 5/14; A61M 5/00; A61M 5/145

(52) U.S. Cl. CPC A61M 5/1413; A61M 5/14546; A61M 5/007

(57) ABSTRACT

An injection system includes: a syringe having a cylinder to be filled with a chemical liquid, a flange projecting laterally from the cylinder, an end portion through which the chemical liquid passes when the chemical liquid is pushed out, and a syringe screw portion formed on a side opposite to the end portion; an adapter for holding the syringe, the adapter having a receiving portion for receiving the cylinder, a regulating wall for abutting the flange, and an adapter screw portion engaged with the syringe screw portion; and an injection device provided with a holder to which the adapter is to be attached. In this injection system, a tightening direction by the syringe screw portion and the adapter screw portion is set in a direction toward the end portion.

### CROSS-REFERENCE TO RELATED PATENT APPLICATIONS

This application is a U.S. National Phase Application under 35 U.S.C. § 371 of International Patent Application No. PCT/JP2019/047855 filed on Dec. 6, 2019 and claims the benefit of priority to Japanese Patent Application No. 2018-229264, filed Dec. 6, 2018, the contents of both of which are incorporated herein by reference in their entireties. The International Application was published in Japanese on Jun. 11, 2020 as International Publication No. WO2020/116621 under PCT Article 21(2).

### FIELD OF THE INVENTION

The present invention relates to a syringe filled with a chemical liquid, an adapter for holding the syringe, and an injection system including the syringe and the adapter.

### BACKGROUND OF THE INVENTION

JP2003-38644A discloses a cylinder holder (adapter) for attaching a syringe to an injection head of an automatic injection device of a chemical liquid. The adapter is formed with a flange insertion groove for holding a flange of the syringe.

### CITATION LIST

### Patent Literature

Patent Literature 1: JP2003-38644A

### TECHNICAL PROBLEM

A flange insertion groove described in JP2003-38644A is formed to be thicker than the flange in order to smoothly receive the flange of the syringe. That is, an opening width of the flange insertion groove is set longer than a length of the flange in a thickness direction of the flange. Accordingly, there is a slight gap between the flange mounted on an adapter and an inner surface of the flange insertion groove. Therefore, when a chemical liquid is injected, the syringe slightly moves forward, resulting in an error in a moving distance of a piston of the syringe. Consequently, this hinders accurate calculation of the moving distance of the piston based on a moving distance of a pressing portion of an injection head. As a result, the calculation accuracy of an injection amount of the chemical liquid is lowered.

### SUMMARY OF THE INVENTION

### Solution to Problem

In order to solve the above-mentioned problems, an injection system as an example of the present invention is characterized in that the injection system includes: a syringe having a cylinder to be filled with a chemical liquid, a flange projecting laterally from the cylinder, an end portion through which the chemical liquid passes when the chemical liquid is pushed out, and a syringe screw portion formed on a side opposite to the end portion; an adapter for holding the syringe, the adapter having a receiving portion for receiving the cylinder, a regulating wall for abutting the flange, and an adapter screw portion engaged with the syringe screw portion; and an injection device provided with a holder to which the adapter is to be attached, wherein a tightening direction by the syringe screw portion and the adapter screw portion is set in a direction toward the end portion.

A syringe as another example of the present invention includes a cylinder to be filled with a chemical liquid; a flange projecting laterally from the cylinder; an end portion through which the chemical liquid passes when the chemical liquid is pushed out; and a syringe screw portion formed on a side opposite to the end portion, wherein a tightening direction by the syringe screw portion is set in a direction toward the end portion.

An adapter as another example of the present invention is an adapter for holding a syringe to be filled with a chemical liquid, the adapter comprising: a receiving portion for receiving a cylinder of the syringe; a regulating wall for abutting a flange of the syringe; and an adapter screw portion formed on a side opposite to the receiving portion, wherein a tightening direction by the adapter screw portion is set in a direction toward the receiving portion.

As a result, the flange of the syringe mounted on the adapter comes into close contact with the regulating wall of the adapter. Therefore, it is possible to prevent the syringe from moving forward when the chemical liquid is injected.

Further features of the present invention will become apparent from the description of the following embodiments illustrated exemplarily with reference to the accompanying drawing.

### BRIEF DESCRIPTION OF THE DRAWINGS

FIG. 1 is a schematic perspective view of an injection head.

FIG. 2 is a schematic block diagram of an injection system.

FIG. 3 is a schematic perspective view of an adaptor.

FIG. 4 is a schematic perspective of a syringe.

FIG. 5A is a schematic rear view of the syringe before moving, and FIG. 5B is a schematic rear view of the syringe after moving.

FIG. 6 is a schematic perspective view of the syringe attached to the adapter.

### DETAILED DESCRIPTION OF THE INVENTION

Illustrative embodiments for practicing the present invention will be described in detail below with reference to drawings. It should be noted, however, that the dimensions, materials, shapes, and relative positions of the components described in the following embodiments are arbitrary and can be changed depending on the configuration or various conditions of the device to which the present invention is applied. In addition, unless otherwise specified, the scope of the present invention is not limited to the embodiments specifically described below. Incidentally, in this specification, terms “upward” and “downward” correspond to the upward direction and the downward direction in the gravitational force direction, respectively. Further, the front side corresponds to the side where an end portion 94 of a syringe 90 is located relative to an injection head 2, and the rear side corresponds to the side opposite to the front side.

### Embodiments

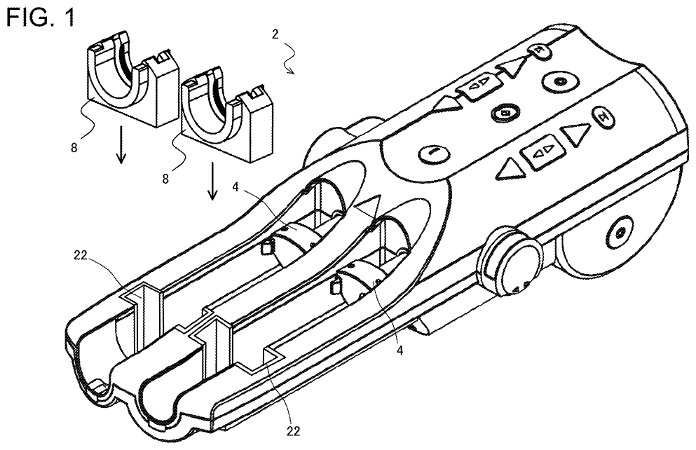

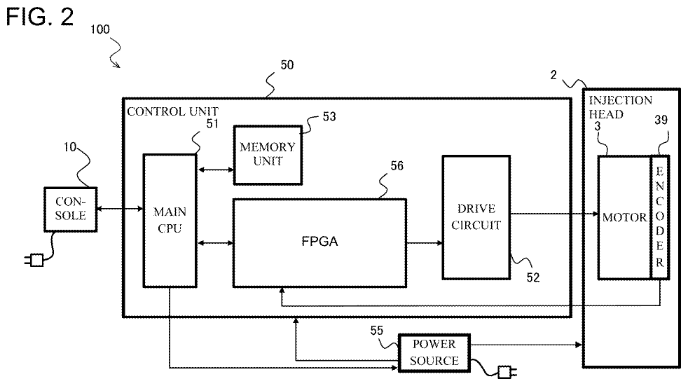

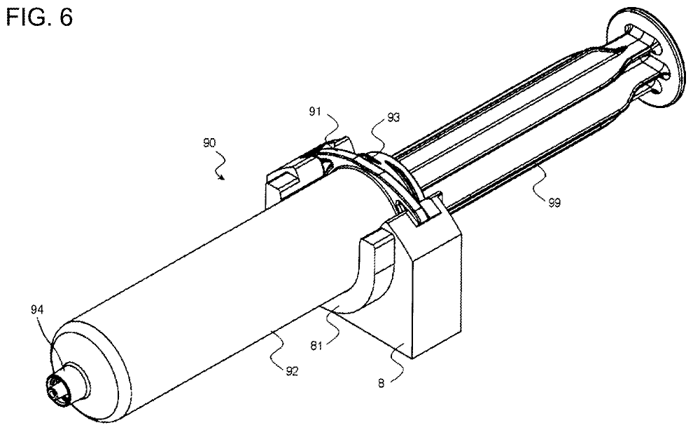

FIG. 1 is a schematic perspective view of an injection device (injection head) 2 for injecting a chemical liquid, and FIG. 2 is a schematic block diagram of an injection system 100. As shown in FIG. 1 and FIG. 2, the injection system 100 for injecting a chemical liquid includes a syringe 90 into which a chemical liquid is loaded, the injection head 2 on which the syringe 90 is mounted, and a pressing portion 4 provided on the injection head 2 and configured to press a rear end of a piston 99 (FIG. 6) inserted into the syringe 90. The injection system 100 also includes an adapter 8 for holding the syringe 90, a holder 22 (FIG. 1) to which the adapter 8 is attached, and a control unit 50 (FIG. 2) for controlling the injection head 2.

The pressing portion 4 is controlled by the control unit 50 such that the pressing portion 4 presses and moves forward the rear end of the piston 99 of the syringe 90 in order to deliver the chemical liquid from the syringe 90. Specifically, the control unit 50 controls a motor 3 included in the injection head 2 such that the pressing portion 4 moves forward when the motor 3 is rotated in a forward direction and the pressing portion 4 moves backward when the motor 3 is rotated in a reverse direction. Further, the injection head 2 includes an adapter 8 for mounting the syringe 90 on the injection head 2. The adapter 8 is attached to a holder 22 of the injection head 2.

The injection system 100 is wired or wirelessly connected to an image capturing device (not shown). At the time of injection of the chemical liquid and at the time of image capturing, various data are transmitted and received between the image capturing device and the injection system 100. Such image capturing devices include, for example, MRI (Magnetic Resonance Imaging) devices, CT (Computed Tomography) devices, angiography devices, PET (Positron Emission Tomography) devices, SPECT (Single Photon Emission Computed Tomography) devices, CT angiography devices, MR angiography devices, ultrasonic diagnostic devices, and vascular imaging devices.

Further, the injection system 100 includes a console 10 having a touch panel as a display unit for displaying an injection state of the chemical liquid, and a control device (not shown) having the control unit 50 and a power source 55. The console 10 and the injection head 2 can be wired or wirelessly connected to each other. The power source 55 may also be provided on the injection head 2 or the console 10, or an independent power source 55 may be provided separately. In addition, the power source 55 may be replaced by a battery.

Further, a remote-control device, such as a hand switch, may be wired or wirelessly connected to the console 10. The remote-control device can also be used to start or stop the chemical liquid injection. Incidentally, the injection head 2 and the control device may be integrally formed with a caster stand (not shown). Alternatively, the injection head 2 and control device may be provided separately and mounted on the caster stand.

In the control device, data of an operation pattern (injection protocol) and data of the chemical liquid are stored in advance. When injecting a chemical liquid into a patient, an operator operates the touch panel of the console 10 to enter an injection speed, an injection amount, an injection time and the patient's physical data such as weight, height, body surface area, heart rate, and cardiac output, as well as the type of the chemical liquid. Then, the control device calculates optimal injection conditions in accordance with the entered data and the data stored in advance. Thereafter, the control device decides the amount of the chemical liquid to be injected into the patient and the injection protocol based on the calculated injection conditions.

Upon deciding the amount of chemical liquid and the injection protocol, the control device causes the touch panel of the console 10 or the head display of the injection head 2 to display predetermined data or graphs. Thus, the operator can confirm the displayed data or graphs. The data of the operation pattern (injection protocol) and the data of the chemical liquid can also be entered from an external storage medium. The control unit 50 of the control device is connected to the motor 3, and an encoder 39 is connected to the motor 3. The encoder 39 transmits a pulse signal having a frequency corresponding to a rotational speed of the motor 3 to the control unit 50.

The pressing portion 4 shown in FIG. 1 has a drive mechanism (not shown). The drive mechanism includes a transmission mechanism connected to a shaft of the motor 3, a ball screw shaft connected to the transmission mechanism, a ball screw nut attached to the ball screw shaft, and an actuator connected to the ball screw nut. The transmission mechanism also has a pinion gear connected to the shaft and a screw gear connected to the ball screw shaft. The transmission mechanism transmits rotations from the motor 3 to the ball screw shaft. Therefore, the rotations of the shaft of the motor 3 are transmitted to the ball screw shaft via the pinion gear and the screw gear. Thus, the ball screw shaft rotates in accordance with the transmitted rotations. The ball screw nut slides in the forward direction or the backward direction in accordance with the rotations of the ball screw shaft. As the ball screw nut slides, the front-end portion of the pressing portion 4 moves forward or backward.

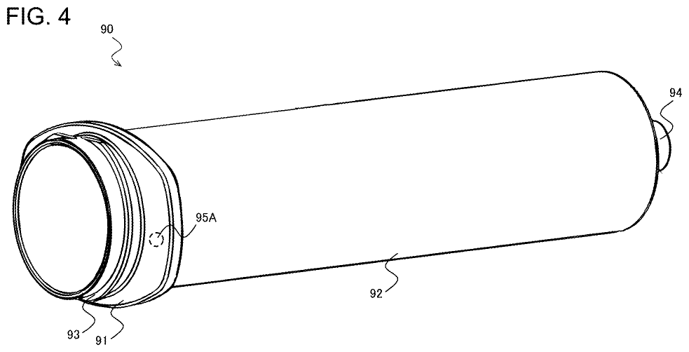

A piston 99 which is slidable in the syringe 90 is attached to the syringe 90. When the motor 3 is rotated in the forward direction in a state where the rear end of the piston 99 abuts the pressing portion 4, the pressing portion 4 pushes the piston 99 forward. As the piston 99 moves forward, the chemical liquid in the syringe 90 is pushed out through the end portion 94 (FIG. 4) and is injected into the body of the patient via an extension tube connected to the end portion 94. At this time, the control unit 50 calculates a moving distance of the pressing portion 4 based on the pulse signal transmitted from the encoder 39. Then, the control unit 50 can calculate the injection amount of the chemical liquid based on the moving distance of the pressing portion 4. When the motor 3 rotates in the reverse direction, the pressing portion 4 pulls the piston 99 in the retracting direction.

The syringe 90 into which the chemical liquid has been loaded may be a prefill syringe. Also, the chemical liquid may be manually loaded into the syringe 90 or may be loaded into the syringe 90 by the injection system 100 or a loading device. In addition, the syringes 90 may be provided with a data carrier such as a RFID or bar code. In the data carrier, information of the loaded chemical liquid is recorded. The injection system 100 can read the recorded information from the data carrier via the injection head 2 to control the injection amount of the chemical liquid. For example, the control device may calculate an optimal injection amount per body weight based on the read information (iodine amount) of the chemical liquid and display it on the touch panel of the console 10.

When injecting the chemical liquid, the operator turns on the power of the injection system 100 and mounts the syringe 90 on the injection head 2. Thereafter, the operator presses the injection button displayed on the touch panel. If the injection head 2 is provided with an operation panel, the operator may press an injection button on the operation panel. Furthermore, the operator may initiate the injection by pressing a button on the hand switch. Alternatively, the operator may turn on the power of the injection system 100 after mounting the syringe 90.

When the injection button is pressed, the control unit 50 transmits a forward rotation signal as a drive voltage to the motor 3. When the shaft of the motor 3 rotates in the forward direction in response to the forward rotation signal, the encoder 39 detects the rotation and transmits a pulse signal to the control device. Thereafter, when the injection is completed and the syringe 90 is removed, the control unit 50 transmits a reverse rotation signal as a driving voltage to the motor 3 in order to move the piston 99 backward. The shaft of the motor 3 rotates in the reverse direction in response to the reverse rotation signal.

The control unit 50 has a memory unit 53 as a storage unit, and the injection protocol is stored in advance in the memory unit 53. The injection of the chemical liquid is performed automatically in accordance with the injection protocol. The injection protocol includes, for example, the injection time, the injection speed, the injection amount, and the injection pressure limit value. The contents of the injection protocol are displayed on the console 10 so that the operator can look at the console 10 to confirm the contents of the injection protocol. The control unit 50 also controls the injection time by using a timer (not shown) and monitors the injection state such as the injection pressure of the chemical liquid. Incidentally, a storage medium in which the injection protocol is stored may be connected to the control device, and the chemical liquid may be injected in accordance with the injection protocol read from the storage medium.

As shown in FIG. 2, the control unit 50 controls the motor 3 and the power source 55 supplies the electric power to the control unit 50 and the injection head 2. The main CPU (Central Processing Unit) 51 of the control unit 50 transmits and receives signals to and from the console 10. The main CPU 51 is a one-chip microcomputer, and executes processing operations such as controlling of the motors 3, predetermined calculations, predetermined controlling, and predetermined determinations in accordance with programs stored in advance in the memory unit 53. The memory unit 53 includes a RAM (Random Access Memory) which is a system work memory for operating the main CPU 51, a ROM (Read Only Memory) which stores programs or system software, or a hard disk drive.

The main CPU 51 transmits and receives signals to and from FPGA (Field-Programmable Gate Array) 56. The FPGA 56 is connected to a drive circuit 52 and the drive circuit 52 is connected to the motor 3. A rotor of the motor 3 is connected to the encoder 39 that outputs a pulse signal corresponding to the rotational speed of the motor 3. The encoder 39 outputs the pulse signal to FPGA 56. In addition, the main CPU 51 transmits power control signals to the power source 55 to control the power supplied by the power source 55. Incidentally, the injection system 100 may include a drive device in which the motor 3 and the control unit 50 are integrally provided and the pressing portion 4 may be driven by the drive device. In addition, the control unit 50 may be provided integrally with the console 10.

Adapter and Syringe

The adapter 8 for mounting the syringe 90 on the injection head 2 is attached to the holder 22 of FIG. 1. The adapter 8 is inserted into the holder 22 from an upper side. The syringe 90 may have different outer shapes depending upon manufacturing companies. Therefore, the adapter 8 is used to mount the syringe 90 having a plurality of types of outer shapes on the injection head 2. The adapter 8 is attached to the holder 22 having a shape complementary to the outer shape of the adapter 8. As the syringe 90 is mounted on the attached adapter 8, the syringe 90 is mounted on the injection head 2. Because different adaptors 8 are used depending on the outer shape of the syringe 90, a plurality of types of syringes 90 can be mounted on the injection head 2.

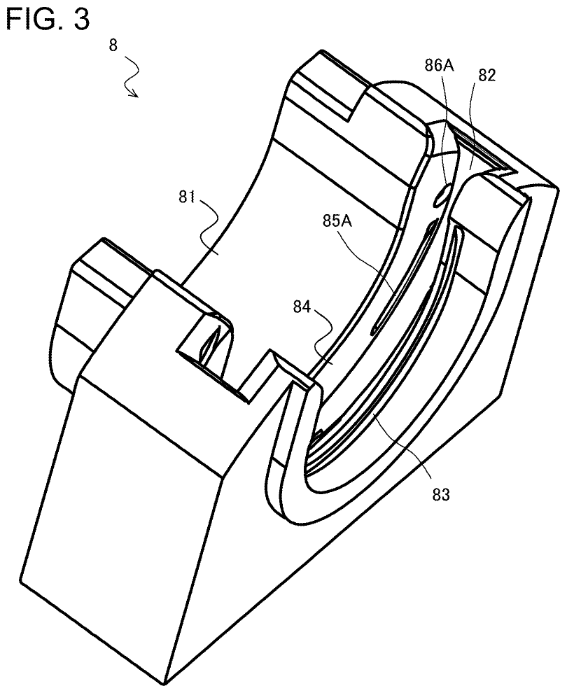

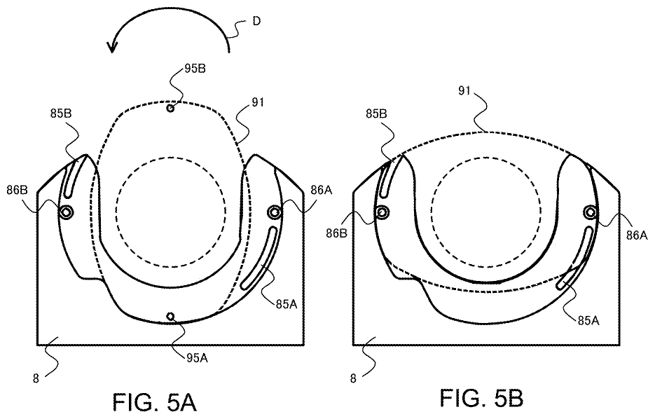

The adaptor 8 and the syringe 90 will be described below with reference to FIG. 3 to FIG. 6. FIG. 3 is a schematic perspective view of the adaptor 8 when viewed from the upper rear, and FIG. 4 is a schematic perspective view of the syringe 90 when viewed from the lateral rear. In addition, FIG. 5A and FIG. 5B are schematic views useful to describe how the syringe 90 is attached to the adapter 8. For illustrative purposes, the rotating syringe 90 is depicted by dotted lines in FIG. 5A and FIG. 5B. FIG. 6 is a schematic perspective view of the syringe 90 attached to the adapter 8 when viewed from the front.

The adapter 8 shown in FIG. 3 has a curved receiving portion 81 for receiving a cylinder 92 of the syringe 90 and a groove 82 which is curved in a substantially U shape and into which a flange 91 of the syringe 90 is inserted. A spiral groove 83 which is an adapter screw portion (threaded groove) is formed in the rear portion of the adapter 8, i.e. on the side opposite to the receiving portion 81. Further, the adaptor 8 has a regulating wall 84 that abuts the flange 91 to regulate the movement of the syringe 90. The adaptor 8 also has a pair of curved grooves 85A and 85B and a pair of concave portions 86A and 86B formed on the inner surface of the groove 82, i.e., on the regulating wall 84. Incidentally, in FIG. 3, only the concave portion 86A is shown.

The syringe 90 shown in FIG. 4 has the cylinder 92, into which the chemical liquid is loaded, and the flange 91 projecting laterally from the cylinder 92. Further, the syringe 90 includes an end portion 94 through which the chemical liquid passes when the chemical liquid is pushed out, and a spiral string 93 which is a syringe screw portion (thread) is formed on a side opposite to the end portion 94 (a rear portion of the syringe 90). The spiral string 93 is engaged with the spiral groove 83. In addition, the syringe 90 has convex portions 95A and 95B formed on the front surface of the flanges 91, i.e., on the surface on an end portion 94 side (only the convex portion 95A is shown in FIG. 4). The convex portion 95A is formed on the front surface of the flange 91 and is shown by the dotted line for convenience of explanation. The syringes 90 also includes the piston 99 (FIG. 6) inserted into the cylinder 92.

When the syringe 90 is mounted on the injection head 2, as shown in FIG. 5A, the syringe 90 before moving (before moving forward) is inserted into the groove 82 of the adapter 8 such that a longitudinal direction of the flange 91 is perpendicular to a longitudinal direction of the adapter 8. Thereafter, the operator rotates the syringe 90 by 90 degrees counterclockwise (in the direction of the arrow D in FIG. 5A) when the syringe 90 is viewed from the rear, and screws the syringe 90 into the adapter 8. That is, the syringe 90 is rotated counterclockwise in the tightening direction (in the moving direction of the syringe 90). Thus, the syringe 90 moves forward while being screwed into the adapter 8. Alternatively, the syringe 90 may be rotated at an angle greater than 90 degrees (e.g., 270 degrees).

At this time, the convex portion 95A formed on the front surface of the flange 91 is received in the curved groove 85A formed on the front side of the groove 82 (regulating wall 84). Then, the convex portion 95A moves in the curved groove 85A, and slides on the regulating wall 84 to the concave portion 86A. Thereafter, the convex portion 95A is received in a concave portion 86A formed on the front side in the groove 82. Here, the curved groove 85A is curved along the outer shape of the flange 91. The concave portion 86A is formed at a position spaced from the curved groove 85A in the extending direction of the curved groove 85A. That is, the concave portion 86A is located on an arc including the curved groove 85A. Further, a step exists between the curved grooves 85A and the concave portion 86A. Therefore, when the convex portion 95A passes over the step, the convex portion 95A collides with the inner surface of the concave portion 86A and collision sound occurs, or the operator can obtain a click feeling. Upon recognizing the collision sound or the click feeling, the operator can confirm that the syringe 90 is properly attached.

Similarly, the convex portion 95B formed on the front surface of the flange 91 is received in the curved groove 85B formed on the front side of the groove 82 (regulating wall 84). Then, the convex portion 95B moves in the curved groove 85B, and slides on the inner surface of the groove 82 to the concave portion 86B. Thereafter, the convex portion 95B proceeds into the concave portion 86B formed in the front side of the groove 82. Here, the curved groove 85B is curved along the outer shape of the flange 91. The concave portion 86B is formed at a position spaced from the curved groove 85B in the extending direction of the curved groove 85B. That is, the concave portion 86B is located on an arc including the curved groove 85B. Further, a step exists between the curved grooves 85B and the concave portions 86B. Therefore, when the convex portion 95B passes over the step, the convex portion 95B collides with the inner surface of the concave portion 86B and collision sound occurs, or the operator can obtain a click feeling. Upon recognizing the collision sound or the click feeling, the operator can confirm that the syringe 90 is properly attached.

The movement and rotation of the syringe 90 are regulated by the front surface of the flange 91 abutting the regulating wall 84. As a result, in a state where the syringe 90 is mounted, the flange 91 abuts the regulating wall 84. Then, as shown in FIG. 5B, the syringe 90 after moving (after moving forward) is held in the adapter 8 such that the longitudinal direction of the flange 91 is parallel to the longitudinal direction of the adapter 8. Further, the convex portions 95A and 95B of the flange 91 are located in the concave portions 86A and 86B.

The syringe 90 moves in the tightening direction as the syringe 90 rotates counterclockwise. That is, the spiral string 93 of the syringe 90 is a spiral protrusion which rotates left in the tightening direction of the syringe 90. The spiral string 93 of the syringe 90 and the spiral groove 83 of the adapter 8 have mutually complementary shapes. Therefore, the spiral groove 83 is a spiral groove that rotates left in the tightening direction of the syringe 90.

Alternatively, the syringe 90 may be moved by rotating the syringe 90 clockwise. In this case, the spiral string 93 of the syringe 90 is a spiral protrusion that rotates clockwise in the tightening direction of the syringe 90. The spiral groove 83 of the adapter 8 is a spiral groove that rotates right in the tightening direction of the syringe 90.

When the syringe 90 is removed from the injection head 2, the syringe 90 is rotated 90 degrees in a clockwise direction, contrary to the case when the syringe is mounted. The syringe 90 is then withdrawn from the grooves 82 of the adapter 8 such that the longitudinal direction of the flanges 91 is perpendicular to the longitudinal direction of the adapter 8, as shown in FIG. 5A.

As described above, the tightening direction of the present embodiment (threading direction) is set to the forward direction. Specifically, the tightening direction by the spiral groove 83 of the adapter 8 is set in a direction toward the receiving portion 81 on which the cylinder 92 is placed. Further, the tightening direction by the spiral string 93 of the syringe 90 is set in a direction toward the end portion 94 of the syringe 90. Therefore, when the syringe 90 is rotated, the front surface of the flange 91 of the syringe 90 is pressed against the regulating wall 84 located between the spiral groove 83 of the adapter 8 and the receiving portion 81. Thus, the syringe 90 is fixed to the adapter 8, and rattling of the syringe 90 is prevented.

FIG. 6 shows the syringe 90 attached to adapters 8. As shown in FIG. 6, the cylinder 92 of the syringe 90 after moving forward projects forward from the receiving portion 81 of the adaptor 8. The piston 99 of the syringe 90 projects rearwardly from the adapter 8. The front end of the piston 99 is inserted into the cylinder 92, and the rear end of the piston 99 is pressed by the pressing portion 4 (FIG. 1). That is, when the syringe 90 is attached to the adapter 8, the piston 99 is located between the cylinder 92 of the syringe 90 and the pressing portion 4. When the pressing portion 4 pushes the rear end of the piston 99, the front end of the piston 99 moves forward in the cylinder 92. As a result, the chemical liquid pushed by the piston 99 is discharged from the end portion 94 of the syringe 90.

According to the syringe 90 and the adapter 8 according to the present embodiment, the flange 91 of the syringe 90 mounted on the adapter 8 is brought into close contact with the regulating wall 84 of the adapter 8. Therefore, it is possible to prevent a gap from being occurred between the flange 91 and the adapter 8. Thus, it is possible to prevent the syringe 90 from slightly moving forward when the chemical liquid is injected. As a result, the moving distance of the pressing portion 4 of the injection head 2 and the injection amount of the chemical liquid can be accurately calculated. Furthermore, rattling of the syringe 90 attached to the adapter 8 can be prevented. Further, because the convex portions 95A and 95B are received into the concave portions 86A and 86B, it is possible to suppress the floating of the syringe 90 during the injection of the chemical liquid.

While the present invention has been described with reference to the respective embodiments, the present invention is not limited to the above-described embodiments. Inventions which are modified within a range not inconsistent with the present invention and inventions equivalent to the present invention are also included in the present invention. In addition, each of the above-described embodiments and each modification can be appropriately combined within a range not contrary to the present invention.

For example, the adapter 8 may be integrally formed with the injection head 2 or may be non-removable from the injection head 2. Further, a pair of concave portions may be formed on the flange 91, and a pair of convex portions corresponding to the pair of concave portions may be formed on the adapter 8. Further, one of the convex portions 95A and 95B may be omitted. That is, one concave portion, one curved groove, and one convex portion may be formed. In this configuration, the curved groove and the concave portion corresponding to the omitted convex portion are also omitted from a pair of curved grooves 85A and 85B and a pair of concave portions 86A and 86B. Further, three or more concave portions, three or more curved grooves, and three or more convex portions may be formed. Further, a convex portion and a concave portion may be formed on the flange 91, and a convex portion and a concave portion corresponding to the convex portion and the concave portion of the flange may be formed on the adapter 8. In this configuration, a curved groove corresponding to the convex portion of the flange 91 may be further formed on the adapter 8. Both the curved groove and the concave portion corresponding to the convex portion may be formed, and one of the curved groove and the concave portion may be omitted.

Further, the adapter 8 may have a step portion which engages with at least a portion of the syringe 90, particularly at least a portion of the flange 91. When the syringe 90 is mounted on the adapter 8 and then rotated, the syringe 90 is fixed at a position where the step portion and at least a part of the syringe 90 are engaged with each other. Thus, the mounted syringe 90 can be stabilized. In addition, the operator can obtain a click feeling at the time of engaging, and can recognize that it is appropriately mounted. As an example, the step portion is constituted by a groove 82 into which the flange 91 is inserted, or by a concave portion or a convex portion formed on the front side (the regulating wall 84) of the groove 82.

In addition, a male thread may be formed as the adaptor screw portion instead of the screw groove, and a screw groove may be formed as the syringe screw portion instead of the male thread. Furthermore, the adapter 8 may have a front part with a receiving portion 81 and a rear part which is separate from the front part and on which the adapter thread is formed. In this configuration, the curved grooves 85A and 85B, and the concave portions 86A and 86B can be formed on the rear end surface of the front portion. Further, the front portion of the adapter 8 may be formed of a material that is harder than the rear portion of the adapter 8. This reduces the possibility that the front portion of the adapter 8 be shaved by the convex portions 95A and 95B of the syringe 90.

### REFERENCE LIST

2: Injection device, 8: Adapter, 22: Holder, 81: Receiving portion, 83: Spiral groove, 84: Regulating wall, 90: Syringe, 91: Flange, 92: Cylinder, 93: Spiral string, 94: End portion, 100: Injection system

1. An injection system comprising:
a syringe having a cylinder to be filled with a chemical liquid, a flange projecting laterally from the cylinder, an end portion through which the chemical liquid passes when the chemical liquid is pushed out, and a syringe screw portion formed on a side opposite to the end portion;
an adapter for holding the syringe, the adapter having a receiving portion for receiving the cylinder, a regulating wall for abutting the flange, and an adapter screw portion engaged with the syringe screw portion; and
an injection device provided with a holder to which the adapter is to be attached,
wherein a tightening direction by the syringe screw portion and the adapter screw portion is set in a direction toward the end portion.

2. The injection system according to claim 1 further comprising:
a convex portion formed on a surface on an end portion side of the flange.

3. The injection system according to claim 2 further comprising:
a concave portion which is formed on the regulating wall so as to receive the convex portion.

4. The injection system according to claim 3 further comprising:
a curved groove formed on the regulating wall,
wherein the concave portion is formed at a position spaced from the curved groove in an extending direction of the curved groove.

5. The injection system according to claim 4, wherein the curved groove is curved along an outer shape of the flange.

6. A syringe comprising:
a cylinder to be filled with a chemical liquid;
a flange projecting laterally from the cylinder;
an end portion through which the chemical liquid passes when the chemical liquid is pushed out; and
a syringe screw portion formed on a side opposite to the end portion,
wherein a tightening direction by the syringe screw portion is set in a direction toward the end portion.

7. The syringe according to claim 6 further comprising:
a convex portion formed on a surface on an end portion side of the flange.

8. An adapter for holding a syringe to be filled with a chemical liquid, the adapter comprising:
a receiving portion for receiving a cylinder of the syringe;
a regulating wall for abutting a flange of the syringe; and
an adapter screw portion formed on a side opposite to the receiving portion,
wherein a tightening direction by the adapter screw portion is set in a direction toward the receiving portion.

9. The adapter according to claim 8 further comprising:
a concave portion which is formed on the regulating wall.

10. The adapter according to claim 9 further comprising:
a curved groove formed on the regulating wall,
wherein the concave portion is formed at a position spaced from the curved groove in an extending direction of the curved groove.
